(19) United States
(12) Patent Application Publication
(10) Pub. No.: US 2007/0270911 A1
(43) Pub. Date: Nov. 22, 2007

(54) HEART STIMULATOR

(76) Inventors: Thomas Doerr, Berlin (DE); Frank Becker, Berlin (DE); Torsten Radtke, Berlin (DE)

Correspondence Address: DALINA LAW GROUP, P.C., 7910 IVANHOE AVE. #325, LA JOLLA, CA, 92037, US

(21) Appl. No.: 11/694,224

(22) Filed: Mar. 30, 2007

(30) Foreign Application Priority Data
May 19, 2006 (DE) 10 2006 023 517.7

(51) Int. Cl. A61N 1/39

(52) U.S. Cl. 607/5

(57) ABSTRACT

A heart stimulator (10) with stimulation unit (1,3,5) connected to stimulation electrode and comprising high voltage capacitor (5), detector (2) for processing physiological signals and for detecting acute ventricular tachycardia, and control unit (4) connected to detector (2) and stimulation unit (1,3,5) designed to to emit stimulation pulses, wherein output signal of detector (2) is tachycardia signal for stable cardiac rhythm with frequency above predetermined tachycardia detection limit, control unit (4) is designed to actuate stimulation unit (1,3,5) to emit a train of stimulation pulses forming antitachycardic therapy in response to a tachycardia signal, and to initiate charging of high voltage capacitor (5), and detector (2) designed to process signals received during charging and generate a tachycardia end signal in the case of frequency below a predetermined tachycardia redetection limit, wherein control unit (4) interrupts charging of high voltage capacitor (5) immediately in response to a tachycardia end signal.

This application takes priority from German Patent Application DE 10 2006 023 517.7 filed 19 May 2006, the specification of which is hereby incorporated herein by reference.

### BACKGROUND OF THE INVENTION

1. Field of the Invention

The invention relates to a heart stimulator with a stimulation unit which is or can be connected to a stimulation electrode for stimulating a ventricle of a heart. The stimulation unit is designed to generate both stimulation pulses and defibrillation shocks, and for this purpose has at least one high voltage capacitor in which the electricity required for a defibrillation shock can be stored. The hear stimulator also has a detector which is designed to process physiological signals received from the heart, and on the basis of these signals detect the presence of an acute ventricular tachycardia or fibrillation. A further component of the heart stimulator is a control unit which is connected to the detector and the stimulation unit and which is designed to respond to an output signal of the detector and to actuate the stimulation unit either to emit a train of stimulation pulses forming an antitachycardic therapy or a defibrillation shock.

2. Description of the Related Art

Such heart stimulators are known in principle and are also referred to as implantable cardioverter defibrillators (ICD's). The heart stimulators referred to here are therefore primarily implantable heart therapy devices which are capable of treating a hear tachycardia.

Within the scope of this application tachycardias are understood to mean both tachycardias in the narrower sense, which are characterised by a stable cardiac rhythm with a pathologically high frequency, and fibrillations. Known therapies, which can be provided by a heart stimulator of the type already mentioned, are antitachycardic stimulation or defibrillation shock.

A defibrillation shock is an electric current surge which is transmitted to the heart and which has a sufficiently high voltage and energy to fully excite a ventricle affected by fibrillation and therefore to render it refractory. Circulating excitations typical of fibrillations are interrupted in this manner. In the case of a tachycardia in the narrower sense, which is often also referred to as ventricular tachycardia, if the ventricle is affected, and which is abbreviated to VT (as opposed to ventricular fibrillation VF), successful therapy by means of antitachycardic stimulation (anti tachyarrhythmia pacing; ATP) is often possible. In antitachycardic stimulation the heart stimulator emits a series of stimulation pulses whose energy is much lower than the energy of a defibrillation shock and which are not painful either. In antitachycardic stimulation such stimulation pulses of comparatively lower energy are emitted with a frequency that exceeds the frequency of the recorded tachycardia. In many cases a tachycardia can be terminated in this manner without the patient suffering pains or without the energy requirement being particularly high.

Since success with an antitachycardic stimulation is not always achieved, it may be necessary to give a defibrillation shock following unsuccessful antitachycardic stimulation.

For example, U.S. Pat. No. 6,718,204 discloses a method for giving such antitachycardic stimulation after a tachycardia is detected, during or shortly before a high voltage capacitor is charged, so that defibrillation shock can be given after the tachycardic stimulation if necessary.

Here there are the contradictory requirements of wanting to save as much as possible on the one hand, i.e. avoiding charging the high voltage capacitor wherever possible, and on the other hand of being able to give a defibrillation shock as soon as possible after an unsuccessful antitachycardic stimulation, which in principle presupposes a capacitor that is already charged.

### SUMMARY OF THE INVENTION

The object of the invention is to find the optimum possible solution to this dilemma.

According to the invention the object is achieved in that a—preferably implantable—heart stimulator of the type already mentioned is designed initially to trigger an antitachycardic stimulation after a fast, monomorphous ventricular tachycardia with a frequency above a fibrillation detection limit is detected, in order to initiate the charging of the high voltage capacitor immediately after the end of the train of stimulation pulses which form the antitachycardic stimulation therapy, and to determine whether the antitachycardic stimulation therapy was successful or not during charging of the high voltage capacitor by evaluating physiological signals received in the heart. If the evaluation shows that the antitachycardic stimulation therapy was successful, charging of the high voltage capacitor is interrupted immediately so that not all the energy required for complete charging of the capacitor is lost. On the other hand, if the evaluation of the physiological signals received after the antitachycardic stimulation therapy is given shows that the tachycardia to be treated is persisting, charging of the high voltage capacitor is continued and finally a defibrillation shock is given. This means that charging of the high voltage capacitor cannot commence until the evaluation of the physiological signal received in the heart indicates that the tachycardia persists. This enables the defibrillation shock to be given as soon as possible.

The heart stimulator is preferably designed to detect at least two types of tachycardias and to associate different consequences with the detection of the particular type of tachycardia. In the case of such a preferred heart stimulator only the detection of a stable tachycardia with a frequency in the range corresponding to a fibrillation causes the control unit initially to trigger an antitachycardic stimulation in order to initiate charging of the high voltage capacitor for the defribrillation after the antitachycardic stimulation is terminated, simultaneously with the beginning (and not until the results are available) of the success check. This therapy sequence is triggered by a signal which is referred to hear as the tachycardia signal. In the case of a tachycardia with a lower frequency (below the fibrillation detection limit), the detector generates another signal which causes the control unit to initiate an antitachycardic stimulation but not the charging of the high voltage capacitor for defibrillation after it is terminated.

In this sense the detector responds to cardiac frequencies of between 150 and 300 beats a minute as the fibrillation detection (corresponding to heart intervals of between 400 ms and 200 ms) so that the control unit initiates the sequence of antitachycardic stimulation followed by immediate charging of the high voltage capacitor, according to the invention, whilst the detector responds to cardiac frequencies of between 100 and 150 beats a minute or heart intervals of between 600 ms and 400 ms, so that it causes the control unit to initiate only an antitachycardic stimulation, but no subsequent charging of the high voltage capacitor.

It is advantageous here for the detector, at least for the first of the two above-mentioned cases, to be designed to record the stability of the tachycardic cardiac rhythm based on an x from y criterion, in the sense that a stable tachyarrhythmia is detected when at least X ventricular intervals within the last Y successive ventricular intervals are shorter than the predetermined fibrillation limit. Suitable values for X are values of between 6 and 30, and suitable values for Y are values of between 8 and 31.

According to the invention the detector of the heart stimulator is designed to detect a stable cardiac rhythm with a frequency above a predetermined fibrillation detection limit, then to generate an output signal which causes the control unit to actuate the stimulation unit to emit a train of stimulation pulses forming an antitachycardic therapy. The control unit is designed to initiate the charging of the high voltage capacitor immediately following the train of stimulation pulses forming the antitachycardic therapy, whilst at the same time—i.e. also immediately after emitting the train of stimulation pulses forming an antitachycardic therapy—the detector analyses physiological signals received from the heart to determine whether the antitachycardic therapy was successful or not. If the detector then generates an output signal which characteristics a successful antitachycardic therapy, the control unit responds to this signal and interrupts charging of the high voltage capacitor immediately. The detection limit at which the detector emits an output signal which causes the control unit to initiate the antitachycardic therapy may be referred to as the tachycardia detection limit or as the fibrillation detection limit, and corresponds to a heart frequency which is normally associated with a fibrillation. In addition to the frequency criterion, the detector uses s a stability criterion when evaluating the physiological signals deriving from the heart, and generates the output signal which causes the control unit to initiate an antitachycardic therapy only when the cardiac rhythm is stable. The control signal emitted by the detector is referred to here as the tachycardia signal. Under certain circumstances this tachycardia signal may be a signal other than the output signal, which the detector generates when it records a raised stable heart frequency which, although associated with a tachycardia, has not yet exceeded the frequency limit for fibrillation.

The output signal which the detector generates after detecting a successful antitachycardic therapy, and which causes the control unit to interrupt the charging of the high voltage capacitor, is referred to for the purposes of this application as the tachycardia end signal.

This tachycardia end signal is generated by the detector according to a preferred design variant of the invention when, after termination of the antitachycardic stimulation, preferably 3 out of 4 consecutive ventricular intervals detected are longer than a predetermined tachycardia detection limit. A preferred value for the tachycardia detection limit is 600 ms.

According to this preferred design variant the heart stimulator emits a defibrillation shock if the control unit has received no tachycardia end signal from the detector by the end of charging of the high voltage capacitor. The defibrillation shock is therefore emitted immediately after the end of charging of the high voltage capacitor if the charging of the high voltage capacitor is not previously interrupted in response to a tachycardia end signal.

In an alternative design variant the detector is designed to generate a tachycardia redetection signal if unsuccessful therapy is detected after antitachycardic therapy, which signal causes the control unit to continue charging the high voltage capacitor and finally initiate the defibrillation shock.

For evaluating the physiological signals deriving from the heart after the antitachycardic therapy the detector compares the cardiac frequency then detected with a frequency value predetermined by a tachycardia redetection limit. This frequency value preferably conforms to the tachycardia and/or fibrillation detection limit.

The physiological to be evaluated by the detector and deriving from the heart is preferably an intracardial electrocardiogram. For recording such an intracardial electrocardiogram the detector is preferably connected to a sensing electrode, or can be connected to such a sensing electrode. A suitable sensing electrode may in this case also be a stimulation electrode.

The stimulation unit of the heart stimulator is preferably connected to at least two different types of ventricular electrodes, namely to at least one ventricular stimulation electrode and to at least one ventricular defibrillation electrode, which has a larger surface than the stimulation electrode. The stimulation electrode is preferably a tip or ring electrode, whilst the defibrillation electrode is preferably a coil electrode arranged a short distance away from a distal electrode cable end.

The antitachycardic stimulation may have the following characteristics in preferred design variants. The number of the train of stimulation pulses forming the antitachycardic therapy is preferably a predetermined value between 1 and 10. The interval between individual stimuli of the train of stimulation pulses and between the last detected ventricular event and the first stimulus of the train of stimulation pulses is preferably set to a duration from 75% to 90% of either the last detected ventricular interval or the average interval between a predetermined number of last recorded consecutive ventricular events. Alternatively, the train of stimulation pulses may have a rising rate at which an interval between two consecutive stimuli is shortened from initially 350 ms to 200 ms, from interval to interval, by a value of between 40 ms and 5 ms.

### BRIEF DESCRIPTION OF THE DRAWINGS

The invention will now be explained in greater detail by way of an exemplary embodiment with reference and associated drawings, in which:

FIG. 1 shows an implantable heart stimulator.

### DETAILED DESCRIPTION OF THE INVENTION

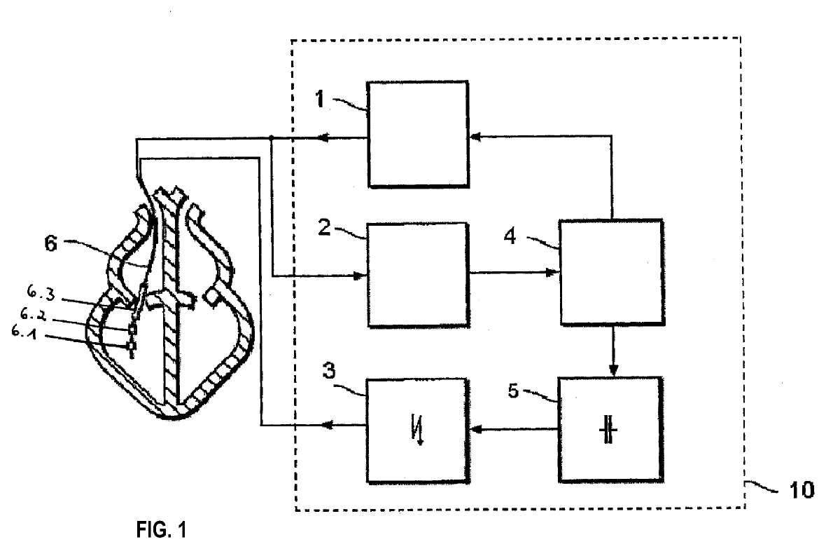

For this purpose FIG. 1 shows an implantable heart stimulator 10, which is connected by an electrode cable 6 to the right ventricle of a heart. Electrode cable 6 carries a ventricular tip electrode 6.1 and a ventricular ring electrode 6.2, together with a shock coil 6.3, for emitting a defibrillation shock. The ventricular tip and ring electrodes 6.1 and 6.2 serve as sensing electrodes. Ventricular tip electrode 6.1 also serves as a stimulation electrode. Ventricular tip electrode 6.1 and ventricular ring electrode 6.2 are connected by electrode cable 6 to a detector 2 which amplifies the electrical potential received, via electrodes 6. and 6.2, by means of suitable input amplifiers, thereby generating an intracardial electrocardiogram. Detector 2 is designed to derive a current cardiac frequency from this intracardial electrocardiogram signal and to compare it with predetermined frequency limits. One of these predetermined frequency limits is referred to within the scope of this application as the tachycardia detection limit or fibrillation limit, and corresponds to a cardiac frequency from which we normally speak about fibrillation of the heart. Detector 2 is also designed to analyse the stability of the recorded cardiac frequency and only generate a tachycardia detection signal when the evaluation of the intracardial electrocardiogram indicates that the acute cardiac frequency exceeds the tachycardia detection limit and also fluctuates so little that it conforms to a predetermined stability criterion.

Detector 2 is connected to a control unit 4, which responds to a corresponding output signal of detector 2.

Control 4 is in turn connected on the output side to a stimulation unit which comprises on the one hand a low energy stimulation unit 1 and on the other a defibrillation unit with a high voltage capacitor 5 and a shock synchronisation unit 3.

Low energy stimulation unit 1 is connected to ventricular tip electrode 6.1 and is designed to emit stimulation pulses of low energy and a maximum voltage of less than 10 volts via ventricular tip electrode 6.1 to a heart. In particular, low energy stimulation unit 1, actuated by control unit 4, is designed to emit a fast train of stimulation pulses as antitachycardic therapy with a train frequency of the stimulation pulses which exceeds the acute cardiac frequency detected by detector 2. Such a train of stimulation pulses is then emitted by low energy stimulation unit 1 when the detector generates a tachycardia detection signal after detecting the conditions previously explained and has emitted it to control unit 4. Control unit 4 is designed to initiate charging of high voltage capacitor 5 immediately after the end of a train of stimulation pulses forming the antitachycardic therapy.

Detector 2 is designed to evaluate the intracardial electrocardiogram recorded immediately after the end of the antitachycardic therapy to determine whether the antitachycardic therapy was successful and the tachycardia was terminated as a result. For this purpose the detector compares a cardiac frequency derived from the intracardial electrocardiogram after the antitachycardic therapy is given with a frequency limit value which is here referred to as the tachycardia detection limit and corresponds to a value which marks the limit between a fast cardiac rhythm not requiring treatment and a low frequency tachycardia with a cardiac cycle length of 600 ms and less. Accordingly this tachycardia detection limit, in the exemplary embodiment, is 100 beats a minute. If the cardiac frequency recorded after the antitachycardic therapy is given is lower than the tachycardia detection limit, detector 2 generates a tachycardia end signal.

Control unit 4 responds to this tachycardia end signal and interrupts the charging of high voltage capacitor 5 already begun immediately. If no tachycardia end is detected until the high voltage capacitor is fully charged, i.e. until the end of charging, and therefore no tachycardia end signal is generated either, charging of high voltage capacitor 5 is continued until the end of charging and shock synchronisation unit 3 is caused to emit the defibrillation shock, synchronised if possible. The defribrillation shock is preferably synchronised to a ventricular hear signal (R-line) or to a ventricular stimulus. The shock is only emitted unsynchronised if (in the case of a shock without confirmation) no ventricular event occurs within 2 s after the end of charging the high voltage capacitor.

Alternatively the control unit could be designed to make the emission of the defibrillation shock after the end of charging additionally dependent on the current cardiac frequency then exceeding a tachycardia redetection limit. If in this case the evaluation of the intracardial electrocardiogram recorded after the end of charging of the high voltage capacitor by detector 2 indicates that the cardiac frequency lies above the tachycardia redetection limit, detector 2 generates a tachycardia redetection signal which causes control unit 4 to actuate shock synchronisation unit 3 so that it emits a defribrillation shock hat is synchronised if possible.

This enables heart stimulator 10 to terminate a monomorphous ventricular tachycardia of high frequency, if possible with an antitachycardic therapy, and to initiate a defibrillation shock as quickly as possible if the antitachycardic therapy is unsuccessful, without too much energy for charging a high voltage capacitor being lost in the case of a successful antitachycardic therapy.

In addition to the features described above, the heart pacemaker may have all the usual features of implantable heart pacemakers or cardioverters/defibrillators of prior art, for example a battery, a telemetry unit for transferring data to a service centre or to an external device such as a patient device, an atrial stimulation unit, an atrial defibrillation unit, an atrial sensing unit, a left ventricular stimulation and/or sensing unit, etc.

What is claimed is:

1. A heart stimulator (10) with
a stimulation unit (1, 3, 5) which is connected or can be connected to a stimulation electrode for stimulating a ventricle of a heart, which is configured to generate stimulation pulses and defibrillation shocks, and which has a high voltage capacitor (5) selected from at least one high voltage capacitor for storing electrical energy for a defibrillation shock;
a detector (2), which is configured to process physiological signals received from said heart and detect a presence of an acute ventricular tachycardia or fibrillation;
a control unit (4) which is connected to said detector (2) and said stimulation unit (1, 3, 5), and is configured to respond to an output signal of said detector (2) and to actuate said stimulation unit (1, 3, 5) for emitting a train of stimulation pulses forming an antitachycardic therapy or for emitting said defibrillation shock wherein said output signal of said detector (2) is a tachycardia signal in a case of a physiological signal reproducing a stable cardiac rhythm with a frequency above a predetermined fibrillation detection limit;
said control unit (4) is configured in response to said tachycardia signal of said detector (2), to actuate said stimulation unit (1, 3, 5) for said emitting said train of stimulation pulses forming said antitachycardic therapy, and to initiate charging of said high voltage capacitor (5) immediately following an emission of said train of stimulation pulses forming said antitachycardic therapy wherein said detector (2) is configured to process said physiological signals received from said heart during said charging of said high voltage capacitor (5) and to test for a presence or absence of a tachycardia after an end of said train of stimulation pulses forming said antitachycardic therapy on a basis of at least one predetermined criterion, and to generate a tachycardia end signal if said physiological signal received from said heart indicates an absence of said tachycardia on said basis of said predetermined criterion; and,
wherein said control unit (4) is configured to interrupt said charging of said high voltage capacitor (5) immediately in response to said tachycardia end signal.

2. The heart stimulator according to claim 1, wherein said predetermined criterion is a tachycardia end criterion which characterises said physiological signal received from said heart in said absence of said tachycardia, and in that said detector is configured to generate said tachycardia end signal if said tachycardia end criterion is met.

3. The heart stimulator according to claim 2, wherein said tachycardia end criterion is met when said physiological signal received from said heart indicates that a predetermined number of consecutive ventricular intervals is longer than a predetermined tachycardia detection limit.

4. The heart stimulator according to claim 3, wherein said predetermined tachycardia detection limit has a value of between 400 ms and 600 ms.

5. The heart stimulator according to claim 3, wherein said predetermined number of ventricular intervals is 3 intervals within 4 consecutive intervals.

6. The heart stimulator according to one of claims 1, wherein said control unit (4) is configured to initiate said emission of said defibrillation shock if said control unit (4) has received no tachycardia end signal by an end of said charging time for said high voltage capacitor.

7. The heart stimulator according to claim 1, wherein said predetermined criterion is a tachycardia redetection criterion which characterises said physiological signal received from said heart in said presence of said tachycardia, and in that said detector is configured to generate said tachycardia end signal if said tachycardia redetection criterion is not met.

8. The heart stimulator according to claim 7, wherein said detector (2) is configured to process said physiological signals received from said heart during said charging of said high voltage capacitor (5), and to generate a tachycardia redetection signal in case of said physiological signal reproducing a cardiac rhythm with a frequency above a predetermined tachycardia redetection limit, and in that said control unit (4) is configured to actuate said stimulation unit (1, 3, 5) for said emitting said defibrillation shock in response to said tachycardia redetection signal.

9. The heart stimulator according to claim 7, wherein said cardiac frequencies predetermined by said tachycardia detection limit and said tachycardia redetection limit are identical.

10. The heart stimulator according to claim 1, wherein said fibrillation detection limit has a value of between 150/min and 300/min.

11. The heart stimulator according to claim 1, wherein said detector is configured to generate said tachycardia signal when a cardiac frequency of said tachycardia signal received from said heart exceeds said fibrillation detection limit in a number of X cardiac cycles from a predetermined number of Y consecutive cardiac cycles, wherein X is smaller or equal to Y, and X has a value of between 6 and 30 and Y a value of between 8 and 31.

12. The heart stimulator according to claim 1, wherein said stimulation unit comprises a low energy stimulation unit (1) which is configured to emit said stimulation pulses, and a defibrillation unit which comprises said high voltage capacitor (5) and a defibrillation shock synchronising unit (3) and is configured to emit said defibrillation shock.

13. The heart stimulator according to claim 12, wherein said low energy stimulation unit (1) is connected or can be connected to a ventricular tip electrode (6.1).

14. The heart stimulator according to claim 12, wherein said defibrillation unit is connected or can be connected to a coil-shaped defibrillation electrode (6.3).

15. The heart stimulator according to claim 1, wherein the detector (2) has a sensing unit with an input amplifier for amplifying electrical potentials received in said heart.

16. The heart stimulator according to claim 1, wherein said detector (2) is connected or can be connected to a ventricular ring electrode (6.2) and a ventricular tip electrode (6.1).

17. The heart stimulator according to claim 1, wherein said detector (2) has means for deriving a cardiac frequency from an intracardial electrocardiogram signal and a comparator for comparing the derived cardiac frequency with frequency limit values, one cardiac frequency limit value of which is predetermined as said tachycardia detection limit.

18. The heart stimulator according to claim 1, wherein the detector (2) is configured to derive a stability of a cardiac frequency over several cardiac cycles from an intracardial electrocardiogram signal, and to compare it with a stability criterion.
